(19) United States
(12) Patent Application Publication
(10) Pub. No.: US 2025/0182854 A1
(43) Pub. Date: Jun. 5, 2025

(54) METHODS AND SYSTEMS FOR PROTEIN EXPRESSION OPTIMIZATION AND VARIANT GENERATION

(71) Applicant: Geaenzymes Co, San Francisco, CA (US)

(72) Inventors: Michael Dunne, Neuilly-sur-Seine (FR); Leonardo Alvarez, Providencia (CL); Simón Correa, Providencia (CL); Patricio Alegre, Providencia (CL); Francisco LLull, Providencia (CL); Gabriel Orellana, Providencia (CL)

(73) Assignee: Geaenzymes Co, San Francisco, CA (US)

(21) Appl. No.: 18/528,496

(22) Filed: Dec. 4, 2023

(51) Int. Cl. G16B 40/00; G16B 5/20

(52) U.S. Cl. CPC G16B 40/00; G16B 5/20

(57) ABSTRACT

The invention provides a method for maximising the production of recombinant proteins by generating the appropriate DNA, RNA, or protein sequence and/or genetic construct required for optimizing protein expression in the corresponding host organism.

### TECHNICAL FIELD

This invention resides in the interdisciplinary domain of biotechnology, with a specific focus on protein engineering, computational biology, and bioinformatics. It notably encompasses methodologies and systems employing a diverse array of computational techniques for optimizing protein expression and generating protein variants. This invention integrates and leverages the power of various algorithmic approaches, including but not limited to, machine learning models, evolutionary algorithms, deep learning architectures, logits-based algorithms, and statistical modeling techniques.

The technical scope of the invention extends to the utilization of these computational methods for analyzing and optimizing DNA (and in turn RNA), and protein sequences. This includes the employment of machine learning models for predicting protein abundance, evolutionary algorithms for codon optimization, and deep learning models for interpreting complex biological data, such as protein translation efficiency from RNA sequences. The invention also covers logits-based methodologies for the generation of diverse protein variants, maintaining their structural and functional integrity. Additionally, it involves the application of statistical models and data-driven algorithms for improving the accuracy and efficiency of protein expression in various host organisms.

Furthermore, this invention is characterized by its integration of these computational methods into a cohesive system, capable of processing and analyzing biological data to produce optimized genetic constructs. The system is designed to operate on either standard and advanced computing hardware, facilitating rapid and scalable optimization processes.

In essence, the invention represents a significant advancement in protein engineering and computational biology, providing a concrete solution to complex challenges in the field. It broadens the scope of variant exploration in protein engineering and paves the way for new approaches in the optimization of protein expression, leveraging a comprehensive suite of computational algorithms and methodologies.

### BACKGROUND

In the realm of biotechnology, particularly in protein engineering, the optimization of protein expression is a pivotal challenge. The goal is to maximize protein production in various host organisms, which requires an intricate balance of biological and technical factors. Traditional approaches have largely centered on manipulating expression vectors and experimental conditions, and employing codon optimization strategies such as matching the natural codon distributions of the intended host organism. These methods, however, often involve extensive trial and error, making them time-consuming and costly.

The conventional wisdom in molecular biology suggests a linear information flow from DNA to mRNA (transcription) and finally to protein (translation), with the translational efficiency being a critical factor in determining protein yield. Typically, transcription rates are influenced by promoters and other regulatory DNA sequences, while translational efficiency hinges on codon optimization. However, standard optimization processes are not fully understood and can yield inconsistent results.

Recent advancements in machine learning (ML) offer promising alternatives. ML technologies excel in interpreting complex biological data, including spatial and sequential information, making them well-suited for tackling protein expression challenges. For instance, the potential of deep learning models to predict mRNA abundance from DNA sequences, indicates that mRNA levels are influenced by the entire gene regulatory structure rather than isolated coding regions.

Furthermore, ML-based methods have been developed to predict soluble expression, leveraging large datasets of proteins. These approaches, while innovative, often act as proxies for protein overexpression rather than directly predicting total protein expression. There are challenges of model generalization in protein expression prediction, particularly for sequences divergent from training data, underscoring the need for models that can navigate beyond known sequence space.

Up to now, existing methods for expression optimization have not fully accounted for the structural properties of mRNA, which are crucial for effective ribosome attachment and initiation of translation. Additionally, the target protein's structure, which can influence protein yield due to aggregation tendencies, has often been overlooked. The development of machine learning methods that can integrate these factors represents a significant leap forward in improving recombinant protein expression yields.

On the other hand, protein variant design involves mutating a known protein to achieve variants with similar functions but enhanced structural and biochemical attributes. The goal is to maintain the core characteristics of the parent protein while optimizing key features such as thermostability or enzyme activity. The challenge lies in generating viable proteins with improved properties from a vast landscape of potential variants. This complexity necessitates a strategic approach to mutation, leveraging evolutionary insights and avoiding random alterations that could destabilize the protein.

Modern variant generation techniques utilize evolutionary signals for sequence design, with tools like ProteinMPNN and RFDiffusion generating sequences corresponding to predefined folds. Scoring methods such as GEMME, EVE, and TranceptEVE assist in evaluating mutation viability. Despite these advancements, generating a large, diverse variant pool remains essential for maximizing the discovery of optimal variants for lab testing.

In this context, our invention introduces an approach that combines machine learning models and evolutionary algorithms to optimize protein expression and generate diverse protein variants. This method not only considers the traditional factors of codon optimization but also incorporates structural elements of mRNA and the target protein, offering a more holistic solution to the challenges of protein expression in biotechnology.

### BRIEF DESCRIPTION OF THE FIGURES

FIG. 1: flowchart representation of variations of an embodiment of a method incorporating a logits-based algorithm for diverse protein variant generation.

FIG. 2: flowchart representation of variations of an embodiment of a method for predicting protein expression from mRNA sequences.

FIG. 3: Illustrates how a model can be formed of three separate blocks: a UNet module, followed by three double convolutional layers, followed by a series of fully connected layers. The input to the model is a preprocessed 17×128×128 tensor obtained from the DNA sequence of the −30:+90 region around the start codon.

FIG. 4: A) Illustrates how the model has strong predictive power in 14 different series splits, with a mean Spearman p value of 0.779. The mean Pearson correlation and R2 values were 0.780 and 0.601 respectively. B) True versus predicted values for predictions on series set k52_rx7, trained on its complement. On this series, the model achieves Spearman p, Pearson r, and R2 values of 0.819, 0.820, and 0.671 respectively.

FIG. 5: A) Illustrates how the model identifies very low (y<25), very high (y>75), and low or high (y<50 or y>50) sequences with high precision, with mean precision values of 0.789, 0.728, 0.814, and 0.787 respectively; B) In all bins and all series splits, the mean absolute error is low compared with the baseline (grey, defined as what we would expect to see if the model predicted values randomly). The MAE is much smaller for values that are predicted to be extreme.

FIG. 6: flowchart representation of variations of an embodiment of a method for codon optimization through evolutionary algorithms.

FIG. 7: flowchart representation of variations of an embodiment of a method for protein expression prediction using machine learning.

FIG. 8: Illustrates an example of the predicted versus experimental expression of the entire proteomes of three different hosts: S. cerevisiae, E. coli and B. subtilis. The data sets (PaxDB) use aggregated abundance profiles from up to 20 different experimental condition sets each, in order to filter out condition-specific effects and capture a more general relationship between protein sequence and abundance.

FIG. 9: Loop for generation of initial variants. Arrows show the flow of data through the loop, and the factors used to calculate the unnormalized PSSM.

FIG. 10: depiction of embodiments of a system incorporating several methods

FIG. 11: Illustrates the comparison of the predicted versus the measured expression of recombinant-expressed proteins.

FIG. 12: Comparison of predicted probabilities vs. actual observed probabilities for 5000 sequence pairs sampled randomly from B. subtilis, E. coli, and S. cerevisiae. The actual expression of randomly selected pair of sequences s1, s2, were modeled as two normal distributions with μ and σ as predicted by the model, and P(expr(s1)>expr(s2)) was calculated accordingly. The figure compares the predicted probability with the actual difference. The figures to the left compare the actual proportion of expr(s1)>expr(s2) for sequences s1 and s2 in different bands of predicted probability.

### DESCRIPTION OF EMBODIMENTS

The present invention describes method 110, method 120, method 130, method 140 and a system 200 for optimizing protein expression and generating protein variants. The embodiments encompass interdisciplinary applications combining protein engineering with computational modeling, primarily leveraging machine learning and evolutionary algorithms. These methods are designed to enhance the production of recombinant proteins through optimized DNA or protein sequences and genetic constructs, catering to different host organisms.

While this patent application presents specific embodiments related to methods and systems for protein expression optimization and generation of protein variants, it should be understood that these are intended primarily for illustration and example. Experts in the field will recognize that a variety of modifications, adaptations, permutations of steps, and alternative approaches may be employed without straying from the essence and scope of the invention. It is important to acknowledge that the scope of this invention is not strictly limited to the described embodiments, but also includes various other viable implementations and methodologies that adhere to the underlying principles and objectives of the invention as it pertains to protein expression optimization and generation of protein variants.

The invention integrates state-of-the-art computational techniques, including machine learning models and evolutionary algorithms. These methods are adept at interpreting complex biological data, enabling the system 200 to optimize protein expression and generate a broad range of protein variants. This includes the use of tools like deep learning models to predict mRNA abundance from DNA sequences, considering the entire gene regulatory structure.

The invention's core feature is its innovative model architecture, which is adept at predicting protein expression using various machine learning models trained on data from diverse organisms. These models incorporate features like codon usage, UTR sequences, and protein language embeddings. The architecture combines elements like UNet modules, convolutional layers, and fully connected layers, emphasizing efficiency and precision.

The embodiments include approaches for codon optimization using evolutionary algorithms. This technique is critical in achieving efficient protein translation across different prokaryotic organisms. The invention also details a logits-based algorithm for generating diverse protein variants, focusing on maintaining the structural and functional integrity of these variants.

Extensive empirical validation and testing form a part of the invention's methodology. This includes correlation analysis of protein expression predictions, testing proteins of varied size and topology, and validating the models with experimental protein expression values. Such rigorous testing and validation highlight the invention's robustness and reliability.

### Benefits

The methods and systems introduced in this invention significantly improve the efficiency and yield of recombinant protein production. By optimizing the protein expression process, these methods reduce the time and cost typically associated with traditional protein engineering approaches.

The invention's versatile approach allows it to be applied across various host organisms and conditions. This broad applicability is particularly beneficial in the biotechnology industry, where different host organisms are used for protein production.

The integration of machine learning and evolutionary algorithms offers improved predictive capabilities. The system can accurately predict protein abundance and expression levels, which is crucial for optimizing gene sequences and protein production processes in biotechnology.

Traditional methods of protein engineering often rely heavily on trial and error, which can be time-consuming and costly. The computational approaches in this invention minimize this dependency, offering a more efficient and streamlined process.

The logits-based algorithm for protein variant generation represents a significant advancement in the field of protein design. This method enables the rapid production of a diverse array of viable protein variants, addressing gaps in current protein design methodologies.

The system's design and methodologies have substantial commercial and industrial implications, especially in enhancing protein production processes and developing new protein-based products. This can lead to advancements in pharmaceuticals, agriculture, the food industry, and other related fields.

The invention also contributes to the scientific understanding of protein expression and variant generation. The integration of computational modeling with protein engineering presents new opportunities for research and development in these areas.

### Methods for Optimizing Protein Expression and Generating Variants

The invention utilizes structural information, from both DNA (which in turn is transcribed into mRNA) and proteins (amino acid sequences), to improve the yield of recombinant protein expression. The invention also requires information related to the specific production process, such as the species of the host organism (E. coli, S. cerevisiae, P. pastoris, etc.) and process conditions, (e.g. temperature, pH, glucose, etc.), and the expression results of other proteins in these conditions and hosts. The method according to the invention then provides new versions of the DNA sequence, protein sequence and genetic construct (plasmid) to optimize the production of the recombinant protein. The optimized product can comprise signal peptides and solubility tags, among others, if required.

### Methods—Logits-Based Algorithm for Diverse Protein Variant Generation

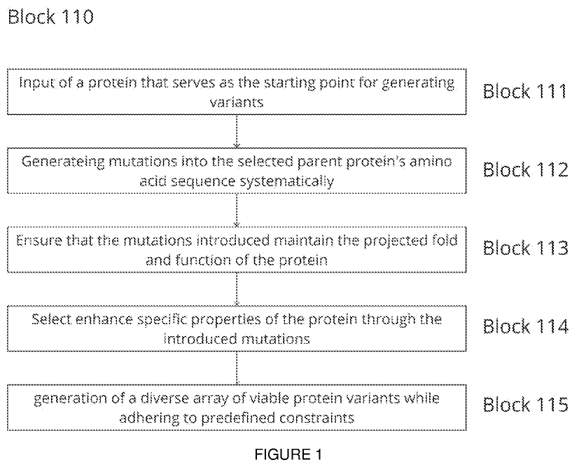

Block S110 recites: a methodology for generating diverse protein variants, starting with a parent protein and systematically introducing mutations to create novel amino acid sequences, maintaining the projected fold and function. Block S110 is composed of the following steps: Block S111: Input of a protein that serves as the starting point for generating variants. Block S112: Generating mutations into the selected parent protein's amino acid sequence systematically. Block S113: Ensure that the mutations introduced maintain the projected fold and function of the protein. Block S114: Enhance specific properties of the protein through the introduced mutations. Block S115: Generation of a diverse array of viable protein variants while adhering to predefined constraints (FIG. 1).

Block S112 leverages advanced probabilistic models, including logits derived from protein-specific large language models such as ProteinMPNN, ESM model family, and other expansive neural network architectures designed for interpreting protein sequences. These models proficiently estimate amino acid occurrence probabilities, thereby facilitating a comprehensive exploration of the protein sequence space. This enables the methodical identification of beneficial mutations and the efficient prediction of sequence functionality. The integrated methodology accounts for a variety of constraints, including but not limited to mutation counts and specific amino acid requirements, providing versatility to accommodate diverse protein sequence analysis applications, scoring methods, and generation techniques. Furthermore, the approach is engineered to interface with an array of sequence generators, expanding its utility to encompass a broad spectrum of protein engineering and design tasks.

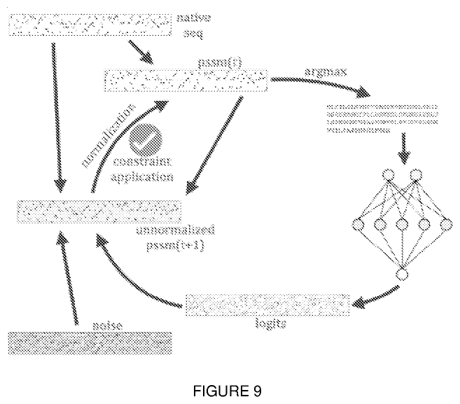

Block S114 details an algorithm for generating a pre-defined number of protein variants from a parent sequence, adhering to specific constraints. The algorithm capitalizes on models outputting logits, necessitating additional inputs depending on the model used. In an example, ProteinMPNN is employed, requiring both a sequence and a protein structure. AlphaFold predicts the structure of the parent sequence, which is then consistently used throughout the algorithm to maintain structural integrity and confine the search space (FIG. 9).

In variations, Block S115 is characterized by its algorithmic framework, which is adept at producing variant sets with high diversity and performance. A key contribution of this methodology is the introduction of a metric to quantify sequence set diversity, an important aspect for ensuring robustness during experimental testing and increasing the likelihood of identifying successful candidates.

Additionally, this logits-based approach is particularly relevant for commercial applications, such as navigating intellectual property challenges, aiding certification processes by minimizing deviations from natural sequences, and providing safeguards against accidental destabilization. This method represents a significant advancement in the field of protein design, offering a fast, efficient solution for generating diverse protein variants tailored to specific requirements.

In examples, the variant generation algorithm of Block S110 operates through a series of epochs, each producing a set of position-specific scoring matrices (PSSMs). These matrices are of dimensions corresponding to the number of seed variants desired, the length of the input sequence, and the amino acid alphabet. The PSSMs start with a uniform encoding of the input sequence and undergo iterative updates at each epoch. These updates integrate three key factors: noise (including normally distributed and peaky noise), logits from a suitable model (e.g. ProteinMPNN), and a bias factor derived from the parent sequence encoding.

Block S110 enhances the diversity of generated sequences while maintaining high identity and score. A diversity metric is employed as a stoppage criterion, selecting the epoch with the highest diversity after stabilization. Subsequently, the number of mutations is adjusted to meet identity constraints, simultaneously improving scores and retaining diversity. This process involves sampling subsets of mutations from the initial variants using pseudo-probabilities as weights.

In variations, a diversity metric for evaluating sequence diversity and likelihood penalizes mutations occurring at the same position, especially if they mutate to the same amino acid. A mutation matrix is formulated, tracking each mutation's occurrence and calculating the diversity based on this matrix. In addition to the diversity metric, two scores are defined to gauge the likelihood of inputs as per the model's logits: the probabilistic score and the magnitude score. The magnitude score, correlating closely with fitness values, is preferred in this context. However, the probability score remains significant due to its robustness against outliers in logistic values.

In variations, the algorithm incorporates dynamic mutation counts and enhanced diversity across multiple runs.

Additionally or alternatively, the algorithm could integrate scores from multiple models and further optimize the generation process by explicitly improving diversity through gradient maximization.

Additionally or alternatively, the variant generation method of block S110 might be based on different exploration and exploitation methods such as reinforcement learning, for example epsilon greedy or any other Q learning algorithm.

Block S110 can also add functional elements to sequences, like signal peptides, promoters, solubility tags, and ribosome binding sites, and expression tags, for further optimization.

Block S110 can deal with sequences from across the domain of life, including eukaryotic species from the genera Trichoderma, Pichia, Saccharomyces, and Aspergillus, and prokaryotic species from the genera Bacillus, Escherichia-Shigella, and Corynebacterium.

### Exemplary Performance: Logits-Based Algorithm for Diverse Protein Variant Generation

In examples, Block S110 leverages ProteinMPNN as a mutation predictor. ProteinMPNN's role in this process is critical, using its logits to calculate a score that acts as a proxy for the fitness of mutants. Its effectiveness was validated through rigorous testing against a deep mutational scanning (DMS) dataset, ensuring reliable performance even in zero-shot applications.

The algorithm has demonstrated high efficiency and diversity in variant discovery under specific constraints. The algorithm was applied to two proteins: GFP (Green Fluorescent Protein) and P84126 (Indole-3-glycerol-phosphate synthase). In the case of GFP, the algorithm was tasked with generating 21 sequences, with specific restrictions on mutations, yielding a diverse array of variants. For P84126, a set of 19 variants was generated. The execution times, leveraging a NVIDIA Geforce RTX 3080, were impressively brief, underscoring the method's rapid processing capability.

The resulting variants from the method were diverse and viable, confirmed by applying metrics based on EVE, ESM-2, and ESM-IF1. Remarkably, the predicted performance of GFP variants outperformed the native sequence in a significant percentage of cases. Similar, though slightly lower, efficacy was observed for P84126 variants.

The algorithm's proficiency in producing quality variants for GFP, a well-studied protein, and P84126, a protein with lower correlation in the DMS test, highlights its versatility and effectiveness. The algorithm's design focuses on initial variant generation through strategic noise addition and guidance towards a fitness landscape minimum. This approach ensures diversity, a critical factor in the process.

In conclusion, the method offers rapid generation of diverse and viable protein variants as it combines the processes of mutating and evaluating candidate sequences. Its two-step approach, combining an initial generation of diverse sequences followed by a targeted search for optimal variants, marks a significant advancement in protein design. The method's ability to produce diverse variants while maintaining structural integrity and desired mutations positions it as a robust tool for protein engineering and biotechnological applications.

Methods—Predicting Protein Expression from mRNA Sequences

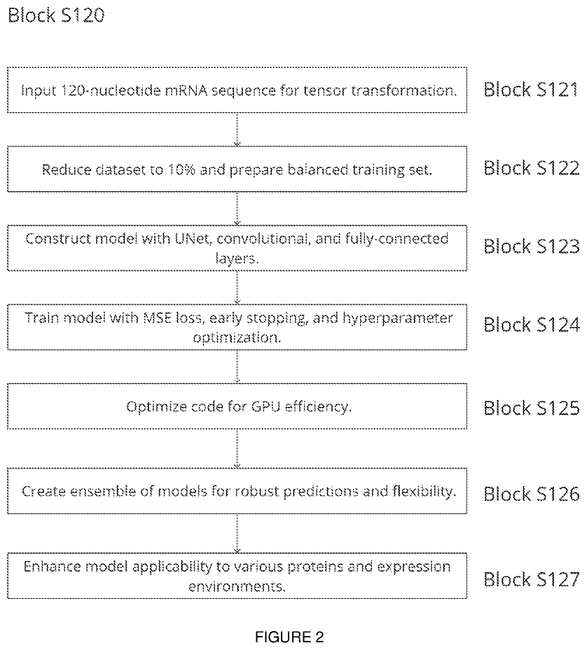

Block S120 recites: Predicting the expression levels of a protein given only the (or equivalently, DNA) nucleotide sequence between positions −30 and +90 relative to the start codon. Block S120 is composed of the following steps: Starting with Block 121, the model requires an input of a 120-nucleotide mRNA sequence spanning from −30 to +90 relative to the start codon, which undergoes preprocessing akin to the UFold method to transform into a tensor for subsequent analysis. The model was trained on a significant dataset of GFP expression measurements, in Block 122, the dataset undergoes a reduction to 10% of its original size, retaining only the first replicate of each sequence to form a balanced training set. The Block 123 describes the model's architecture, beginning with a UNet module and followed by convolutional and fully-connected layers, designed for efficient data processing and prediction. Block 124 outlines the training process, utilizing Mean Squared Error loss and early stopping to avoid overfitting, complemented by hyperparameter optimization to fine-tune performance. To achieve computational efficiency, as described in Block 125, the code is optimized for rapid execution on high-performance GPUs. An ensemble of models, as presented in Block 126, contributes to the robustness of predictions and provides flexibility in application by enabling uncertainty estimates. Finally, Block 127 extends the model's utility, allowing for adjustments in architecture and application to diverse protein constructs and environmental conditions, ensuring the model's adaptability and efficacy in predicting protein expression from mRNA sequences (FIG. 2).

In a variation, Block S120 includes a training dataset derived from Cambray et al.'s study, including luminosity measurements of 244,000 GFP constructs, each featuring a distinct 96-nucleotide leader sequence. The first 90 nucleotides of this sequence, as well as the last 30 nucleotides of 5′ untranslated region (5′UTR), i.e. −30 to +90 relative to the start codon, form the primary input to the model, with the luminosity value serving as the model's output metric.

In variations, Block S120 can include a train/test split methodology designed to prevent the model from recognizing sequence-specific signals in the test set. This is achieved by minimizing sequence similarity between the training/validation and test sets. Notably, each sequence in the source data appears in three variants with minor nucleotide differences ranging from 1 to 4 nucleotides, averaging a mean pairwise difference of 2.35 nucleotides. To ensure data integrity and avoid redundancy, only the first replicate of each sequence is used in the model training and testing process. Furthermore, the dataset is meticulously organized into 56 distinct mutational series. Each series contains several thousand variations of a single seed sequence, with high sequence similarity within a series but distinctiveness across different series. To ensure comprehensive coverage of the sequence space, the dataset is divided into 14 test groups, each encompassing four unique mutational series. The model training process is conducted on sequences from 52 series, while testing is performed on the remaining four series in each group. This strategic arrangement ensures that no sequence in the test set is similar to any sequence in the training or validation sets, thereby enhancing the model's generalizability and predictive accuracy.

In variations, the model's input process involves the preparation of a 120-nucleotide sequence, capturing the region from 30 nucleotides preceding to 90 nucleotides including and following the start codon. This sequence is preprocessed using a methodology akin to the UFold approach, which transforms a sequence of length l into a tensor of dimensions 17×L×L, with l being 120 nucleotides, and L is determined as the smallest multiple of 16 greater than 120, resulting in L being 128. This preprocessing involves executing a Kronecker product between a one-hot-encoded representation of the sequence and itself. The code for this process has been optimized for increased computational efficiency, reducing the execution time from approximately 0.3 seconds on a single CPU to 0.003 seconds, and is designed for effective parallel processing of multiple sequences.

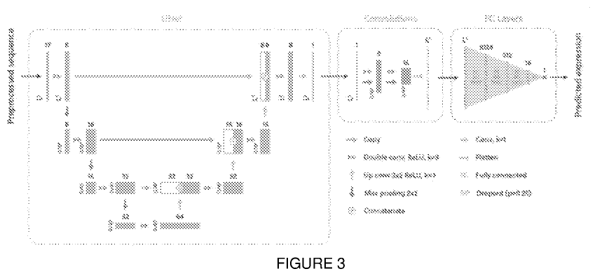

In a specific example of the architecture of the model, it commences with a UNet module, which, while inspired by the UFold method, has been strategically reduced in size to a height of 4. This reduction does not significantly impact the model's performance but rather enhances the speed of training and inference. Following the UNet module are several convolutional layers, which further process the data. The architecture culminates in a series of fully-connected layers, forming a robust structure capable of intricate data analysis and predictive operations. This architecture, illustrated in FIG. 3, represents a balanced integration of complexity and computational efficiency, optimized for the model's specific requirements.

Some variations of the model's training process incorporate specific strategies to efficiently reduce the training time. Initially, the dataset was condensed to 10% of its full size for development purposes. This reduction involved a preliminary filtering step to retain only the first replicate from each sequence. Subsequently, a total of 24,400 data points were sampled to ensure a balanced representation across 99 bins of expression values, each bin being of equal size. The training of the model was conducted using Mean Squared Error (MSE) loss, limited to a maximum of 100 epochs, and included an early stopping mechanism set at 20 epochs to prevent overfitting. The model underwent training thrice on each of the designated series splits. This process involved an extensive hyperparameter optimization, achieved through a grid search across various runs of a single training split, specifically the k52_rx1 split, to maximize the test R2 score. Key training parameters included a batch size of 20, a learning rate of 10e-3, and a weight decay of 10e-5, with a dropout probability of 0.25 applied in the linear layers to enhance generalization. Training was executed on a single AWS NVIDIA Tesla K80 GPU for computational efficiency. In the final stage, the production model was constituted as an ensemble of all 14 models derived from the different series splits. This ensemble approach allows the user to obtain uncertainty estimates: each different model should return slightly different predictions due to natural variation inherent in the data set. The large number of available models also allows for the option to select a subset of models, depending on the specific time and computational constraints of the user, thereby offering flexibility in the model's application.

Variations of the implementation of the model include extending its applicability to diverse protein constructs, adjusting model architecture, utilization of other sequence ranges, adjustments to other expression environments, and/or other information to improve results of the model. As a neural network model, its design focuses on extracting spatial information from the mRNA structure surrounding the start codon. This strategic approach enables the model to predict protein expression levels with remarkable accuracy. Tailored for optimizing gene sequences within a specified protein and expression system, the model has demonstrated its efficacy on a vast dataset of GFP sequence variants.

Block S120 can deal with sequences from across the domain of life, including eukaryotic species from the genera Trichoderma, Pichia, Saccharomyces, and Aspergillus, and prokaryotic species from the genera Bacillus, Escherichia-Shigella, and Corynebacterium.

### Exemplary Execution and Performance: High Correlation Achievement,

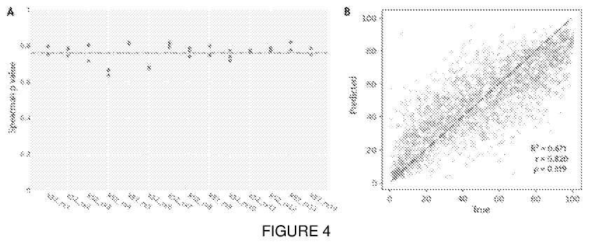

Exemplary execution of the model is demonstrated by its consistent achievement of high correlation values. The model was rigorously trained and tested across 14 distinct series subsets, with each subset undergoing three separate evaluation rounds. The results were consistently robust, showcasing mean Spearman ρ, Pearson r, and R2 values of 0.779, 0.780, and 0.602, respectively. This high degree of correlation underscores the model's robust predictive capability. For each series subset, the model variant that exhibited the highest ρ values on the test set was identified as the most accurate and taken forward for further analytical processes (FIG. 4).

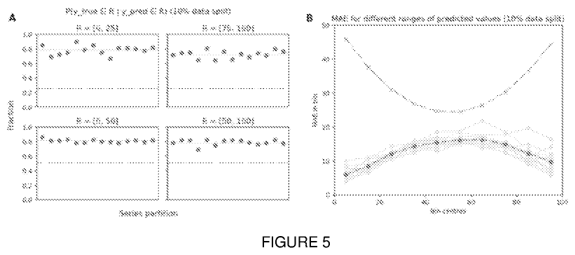

As shown in FIG. 5, the model's precision in categorizing protein expression levels was assessed with remarkable results. On average, across the best-performing models for each series split, the model accurately categorized very low expression sequences (values between 0 and 25) with a precision rate of 78.9%. This is significantly higher than a baseline model, which would randomly predict values between 0 and 100 with a 25% accuracy rate. Additionally, the model demonstrated a precision of 72.8% for very high expression values (75 to 100). For broader classifications (values below 50 and above 50), the model achieved mean precision rates of 81.4% and 78.7%, respectively, outperforming the baseline model's 50% accuracy. These results indicate the model's effective predictive quality, even when considering potential deviations in predicted values.

The model's performance in predicting low and high expression values was characterized by greater accuracy, especially at the extremes of the data range. Across all series runs, the model's predictions for sequences towards the edge of the data range exhibited lower mean absolute errors (MAE). Specifically, for predictions within the range of 0 to 10, the mean MAE across all series was 5.57, and for the range of 90 to 100, it was 9.89. These values are markedly lower compared to the MAE of 16.15 observed in the mid-range region (50 to 60). This contrasts starkly with what would be expected from random predictions, where edge values would typically have a much higher MAE. The model's ability to predict with greater accuracy at these critical ranges is indicative of its refined predictive algorithm and tailored approach to handling expression data. The model's ability to precisely forecast these values hinges on its nuanced understanding of RNA structures. For instance, the model effectively identifies tight and strong RNA structures that cover the ribosome binding site, which are typically associated with lower expression levels. This capacity to discern such intricate RNA structures empowers the model to predict expression levels with a high degree of precision, especially in cases involving extremal values.

The model has been meticulously trained and evaluated on a comprehensive dataset comprising variant GFP sequences. Its training involved sequences distinct from those used in testing, ensuring robustness and reliability in its predictive capabilities. While the model was tested predominantly within the context of GFP expression, there is strong potential for broader applicability. The model possesses the versatility to capture universal characteristics of RNA, making it a promising tool for diverse experimental setups and organisms.

### Methods—Codon Optimization Through Evolutionary Algorithms

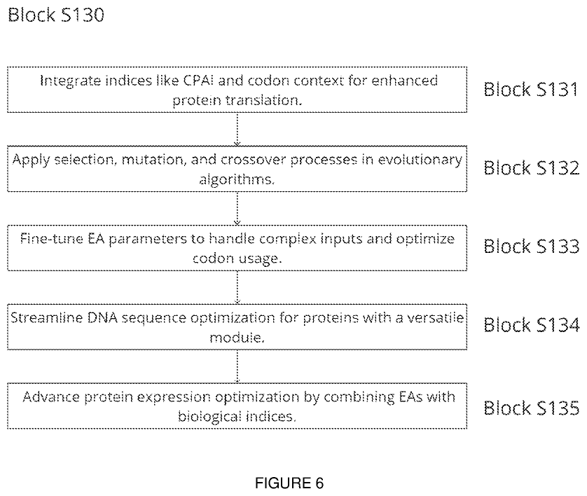

Block S130 recites: a novel approach for optimizing codon usage to enhance protein expression levels across organisms across all domains of life from Archaea, through Prokaryotes to Eukaryotes. Codon optimization is vital for achieving efficient protein translation. Various synonymous DNA sequences can code for the same protein, but not all yield equal translation efficacy. Factors influencing this include RNA secondary structures, homopolymer avoidance, restriction sites, organism-specific GC content, codon bias, and codon context. Utilizing Evolutionary Algorithms (EA), this method significantly improves upon traditional techniques by incorporating comprehensive indices like Codon Pair Adaptation Index (CPAI), a faster implementation of CC (Codon Context) that mainly consider pairs of codons rather than codons individually, also the STOP codon is explicitly considered. Block S130 is composed of the following steps: Block S131 integrates advanced indices such as the Codon Pair Adaptation Index (CPAI) and considers codon context, including the STOP codon, for improved translation efficacy. Block S132 employs evolutionary principles of selection, mutation, and crossover to navigate the complex optimization landscape without learning from past iterations. Block S133 requires meticulous fine-tuning of evolutionary algorithm parameters to effectively address input complexities. Block S134 describes a codon optimization module that streamlines the derivation of optimal DNA sequences, accommodating various applications like fixed sequences and custom cost functions. Finally, Block S135 represents the culmination of the method, showcasing the potential of evolutionary algorithms combined with biological indices to push the frontiers of targeted protein production in diverse organisms (FIG. 6).

Evolutionary Algorithms, inspired by biological evolution, leverage selection, mutation, and crossover processes. Unlike ML models, EAs do not learn from past iterations and require fine-tuning of parameters. Their strength lies in handling complex inputs and providing solutions where derivative-based methods fall short.

In specific examples, a codon optimization module streamlines the process of deriving the optimal DNA sequence for a given protein. This module also extends to translating DNA sequences, dynamic programming-based optimization, restriction site identification, automatic CDS analysis, and mathematical analyses. The module offers a range of applications:
1. mainex: Optimizes the DNA sequence for a single protein, allowing for optional arguments.
2. fixedcodex: Focuses on fixed DNA sequences for certain amino acids, reflecting real-world constraints.
3. customcostfunex: Employs a custom cost function targeting specific optimization goals.
4. bothoptex: Demonstrates simultaneous optimization for two organisms, highlighting the module's versatility.

### Exemplary Execution and Performance: Experimental Validation and Results

Block S130 was experimentally validated using the Green Fluorescent Protein (GFP), due to its quantifiable fluorescence. Notably, no specific restriction sites were avoided in these tests. The results indicate a quadratic-like trend in CPAI values, with significant variation in codon usage across different CPAI targets. This trend confirms theoretical expectations about the distribution and efficacy of varying CPAI values in protein expression.

The method encapsulated in Block S130, represents an advancement in protein expression optimization. By harnessing the power of Evolutionary Algorithms and integrating complex biological indices, it opens new avenues for efficient and targeted protein production in various prokaryotic organisms.

### Methods—Protein Expression Prediction Using Machine Learning

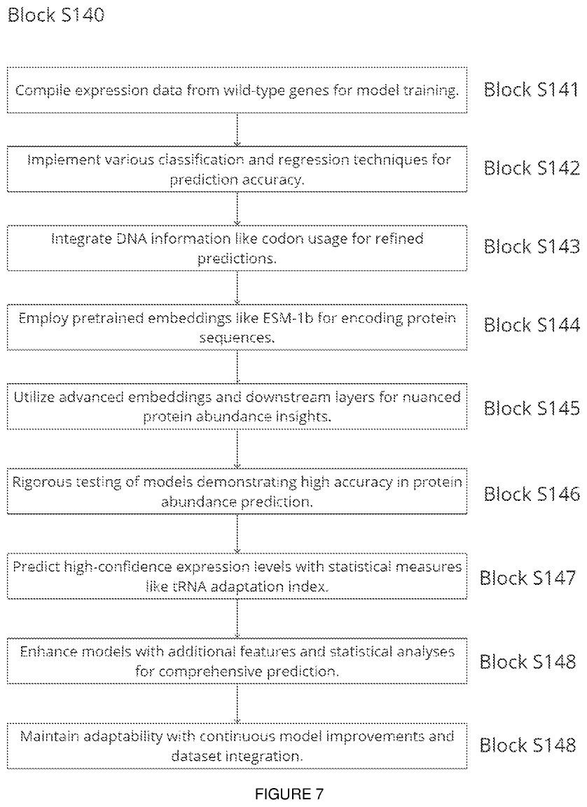

Block S140 recites: method for predicting protein expression using machine learning models. Leveraging amino acid sequences of proteins, these models might be trained on data from various organisms including E. coli and S. cerevisiae or other relevant organisms. The models employ different scenarios, including binary classification and regression tasks, demonstrating remarkable accuracy in predicting protein abundance. Block S140 is composed of the following steps: Block S141 involves compiling a database of expression data from wild-type genes, while Block S142 utilizes a range of predictive processes from binary classification to multi-label regression for accuracy. Block S143 enhances the model by integrating additional DNA information, crucial for developing abundance optimizers. Block S144 uses pretrained embeddings such as ESM-1b for encoding sequences, and Block S145 employs advanced embeddings like ESM-2, UniRep, and BERT, combined with processing layers for in-depth insights. Block S146 sees the models being rigorously evaluated, showcasing high accuracy in abundance prediction, and in Block S147, the method predicts expression levels using statistical measures like the tRNA adaptation index. Block S148 further develops the models by incorporating additional features and statistical analyses, like ANOVA, to identify influential factors, ensuring Block S149 that the method remains adaptable and at the cutting edge of protein abundance prediction technology (FIG. 7).

In variations, Block S140 can include investigating the effects of amino acid sequences on expression levels using a database consisting of expression data for wild-type genes from various species compiled from multiple experimental sources (e.g. PaxDB).

In variations, the models operate under specific constraints, such as a maximum sequence length of 1022 amino acids and an upper limit of 1000 ppm for protein abundance. They incorporate a variety of predictive processes, such as binary classification, a logarithmic regression process, a logarithmic binary classification process, a tertile multi-label classification process, a quartile multi-label classification process, with techniques like logistic regression, Support Vector Regression, MLP classifier, K neighbors classifier, and Random Forest classifier, showing high efficacy.

In advancing the protein abundance prediction models, Block S140 integrates additional DNA information, like codon usage and UTR sequences, to refine and enhance accuracy. This integration is a crucial step towards developing abundance optimizers, capable of recommending optimal coding sequences for given amino acid sequences and organisms.

In specific examples of the architecture of the model, we used the pretrained ESM-1b embedding to encode our amino acid sequences, and then trained a feed-forward fully-connected neural network to predict expression. Protein language embeddings are able to capture implicit information contained in protein amino acid sequences, including aspects of structure, function and chemical properties. They are trained on large databases of natural protein sequences, and convert protein sequences into relatively low dimensional vectors within an abstract embedding space.

Additionally or alternatively, the TAPE (Tasks Assessing Protein Embeddings), ESM-2, UniRep and BERT embeddings can be used. For predicting protein abundance, TAPE embeddings can offer nuanced insights into how the sequence composition might influence expression levels. ESM-2, an evolution of ESM-1b, offers refined predictions by capturing nuanced features in protein sequences. These embeddings, each with unique strengths in interpreting protein language, are combined with downstream processing layers to model the correlation of these features with abundance levels observed in experimental data.

The models in Block S140 are rigorously tested and evaluated, displaying high classification accuracy and R2 values. This performance is a testament to their capability in evaluating protein abundance across different organisms and conditions. The methodology adopted for these models allows them to predict protein abundance not only accurately but also efficiently, with negligible run times in the optimization process.

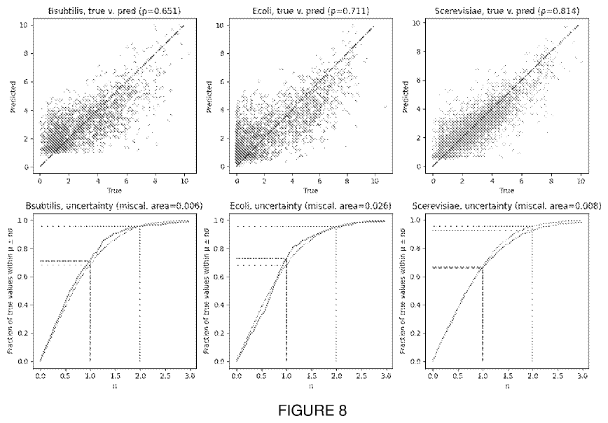

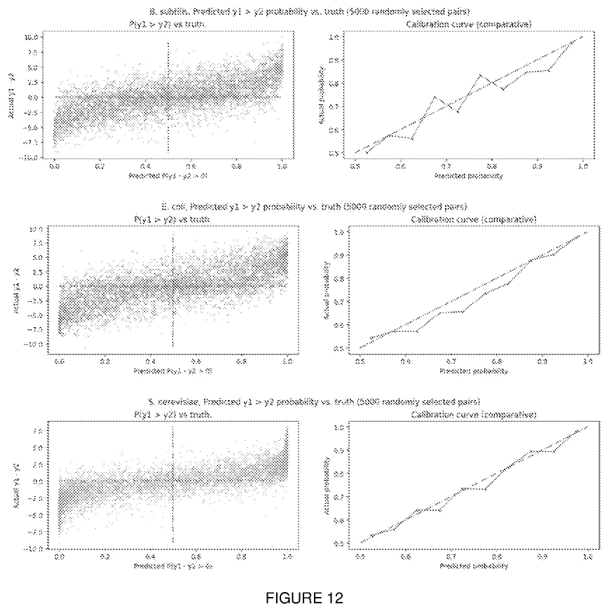

In an example, the method according to the invention is able to predict expression to a high degree of accuracy (Spearman's p=0.68-0.81), in species including S. cerevisiae, B. subtilis, and E. coli. (FIG. 8). In addition the model is able to provide accurate confidence margins, which can be used to identify when the model is more or less sure. When comparing random pairs of sequences from the test sets, the models correctly identify which sequence has higher expression in 81.1%, 74.5%, and 73.2% of cases for S. cerevisiae, E. coli, and B. subtilis (FIG. 12). These percentages rise to 95.5%, 93.9%, and 92.3% when considering pairs of sequences for which the model returns high-confidence (p>0.9) predictions.

In variations, the approach implemented various statistical measures like the tRNA adaptation index (tAI) to correlate with measured abundance. Subsequent stages of model development explored incorporating additional features and techniques. These included the use of embeddings from models like ESM-fold and OmegaFold, as well as exploring the impact of other molecular features such as molecular weight and GC content. The models were also subjected to different statistical analyses, like ANOVA, to identify key factors influencing protein abundance.

As the field evolves, the approach in Block S140 remains adaptable, with continuous improvements and iterations being part of the development cycle. The incorporation of new datasets, exploration of different features, and integration of machine learning techniques ensure that these models stay at the forefront of protein abundance prediction technology. In conclusion, the methods outlined in Block S110 represent a significant advancement in the field of protein engineering and biotechnology. They offer a robust and versatile tool for accurately predicting protein abundance, which is crucial for various applications, including optimizing gene sequences and protein production processes.

Block S140 can deal with sequences from across the domain of life, including eukaryotic species from the genera Trichoderma, Pichia, Saccharomyces, and Aspergillus, and prokaryotic species from the genera Bacillus, Escherichia-Shigella, and Corynebacterium.

### Exemplary Execution and Performance: Empirical Validation and Correlation Analysis of Protein Expression Predictions

Exemplary execution of the model involved validation of the models experimentally, including a selection of different proteins which were expressed recombinantly, and the expression values (in relative fluorescence units) were correlated with predictions from the models. The main goal from these validations is to correctly differentiate between highly and poorly expressed proteins, and decide in a high-throughput manner which sequence variants will be the best suited to be further produced in the corresponding host organism, or under a desired biotechnology platform.

For these validations, proteins of different sizes and topologies were tested, producing them in E. coli, to explore the limitations of these models.

The method according to the invention has shown good correlation with experimental protein expression values, with correlations of:
r (Pearson correlation)=0.789
R² (determination coefficient)=0.637
ρ (Spearman correlation)=0.551

### System

### Integrated Computational Platform for Protein Expression Optimization and Variant Generation

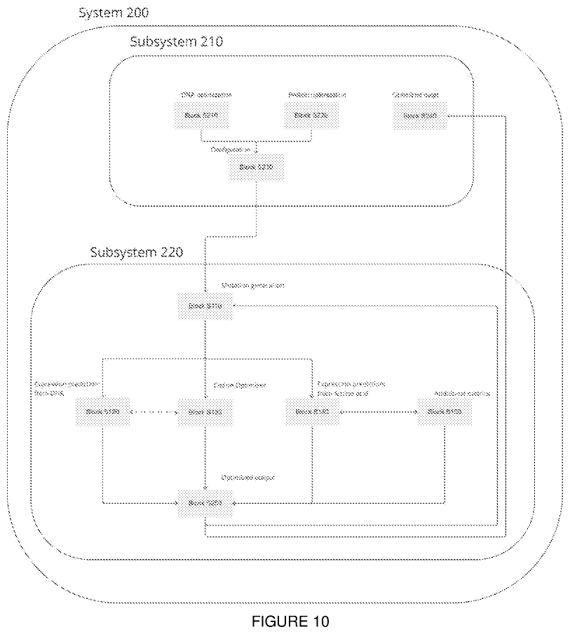

The system 200 outlined in this invention is a comprehensive computational platform designed to optimize protein expression and generate diverse protein variants efficiently. The system integrates machine learning models and evolutionary algorithms to process and analyze protein sequences, aiming to maximize recombinant protein production in various host organisms. This platform is capable of operating on standard computing hardware, including both CPUs and GPUs, enabling rapid prediction and optimization of protein sequences (FIG. 10).

The system/platform 200 includes two interconnected subsystems: the input reception subsystem 210, and the data processing engine subsystem 220.

Subsystem 210 is responsible for receiving input information, either derived from DNA sequences (Block S210) or from protein sequences (Block S220). Subsystem 210 also receives configuration information in Block S230, which may include but is not limited to, codon or nucleotide specification, 5′ UTR sequences, restriction enzyme specifications, amino acid restrictions, number of target mutations, computing target time, and expression features, including the host organism. Block S230 can also receive as input specific operation conditions of the productive process selected from temperature, pH, glucose feeding rate, and substrates. These inputs are vital for the system to understand the specific requirements of the protein expression process in various host organisms. Subsystem 210 then combines all the input parameters and configurations and passes them to subsystem 220 for further processing. Subsystem 210 also incorporates Block S240 that is in charge of receiving and displaying to the user the optimized output.

The data processing engine in subsystem 220, is at the heart of the system and uses the methods described above to predict protein expression levels. This subsystem is in charge of processing the input parameters provided by subsystem 210 and generates an optimized output which is then passed back to subsystem 210. Subsystem 220 utilizes RNA spatial models for expression prediction (Block S120) and species-specific protein abundance models, utilizing amino acid sequences (Block S140). The optimization algorithms in subsystem 220 incorporate both machine learning and evolutionary algorithms: this module optimizes the amino acid sequences and DNA sequences for enhanced expression efficiency. It employs methods like codon optimization through evolutionary algorithms (Block S130) and a logits-based algorithm for diverse protein variant generation (Block S110).

In addition to the methods already described, subsystem 220 can incorporate additional metrics in Block S150, which derive from fine-tuning language models pre-trained with protein sequences or any other machine learning model, and using such fine-tuned models to predict specific target metrics such as stability, solubility, pH stability, enzyme activity, among other metrics. Block S150 takes as an input a data set of protein or DNA sequences and an input protein or DNA sequence and returns a scored protein sequence or DNA sequence for the specific metrics.

The optimized output in Block S250 generates optimized sequences, including the final genetic construct sequence, protein sequence, and optimized DNA sequence. It can also add functional elements like signal peptides, promoters, solubility tags, and ribosome binding sites, and expression tags, for further optimization.

The system 200 is designed for high throughput, capable of processing hundreds of sequences per second. It can be easily scaled for parallel processing, accommodating an arbitrary level of sequence throughput. This makes it significantly faster than traditional experimental methods, providing a practical and efficient solution for protein engineering applications.

Additionally, the system 200 features a user-friendly interface, allowing easy input of data and parameters. It integrates seamlessly with external databases and analysis tools, offering a flexible and adaptable platform for various biotechnological applications. The system is capable of optimizing existing protein sequences uploaded and discovering new proteins that will have the desired function.

Additionally or alternatively, the system has the ability to send optimized DNA constructs directly to service providers (e.g. DNA synthesis companies, protein synthesis companies, protein engineering companies), emphasizing the streamlining of the process from design to synthesis.

The system 200 incorporates multimodal information for optimizing protein expression, including codon frequency, RNA stability, and protein sequence and structure constraints to maintain the protein function while optimizing for certain traits. Thus by its nature the system optimizes both preserving global protein properties while at the same time optimizes aspects specific to RNA structure.

The system 200 can additionally or alternatively incorporate other multimodal information such as protein secretion pathways, among others.

### Exemplary Execution and Performance of the System: Predicted Versus Experimental Expression of Recombinantly Expressed Proteins

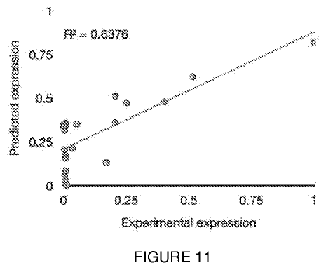

The system's exemplary performance is highlighted by its ability to accurately predict and correlate with experimental protein expression values. The integration of machine learning and evolutionary algorithms ensures a high degree of precision and accuracy, as demonstrated by the correlation coefficients and precision rates. FIG. 11 shows the comparison of the predicted versus the experimentally measured expression of recombinant expressed proteins (normalized).

### CONCLUSIONS

The integrated computational platform 200 represents a significant leap in the field of protein engineering. Its ability to rapidly and accurately predict protein expression, optimize sequences for higher yields, and generate diverse protein variants makes it an invaluable tool in biotechnological research and industrial applications.

1. A method for optimizing protein expression in host organisms, the method comprising:
a. a method incorporating a logits-based algorithm for diverse protein variant generation.
b. a method for predicting protein expression from mRNA sequences.
c. a method for codon optimization through evolutionary algorithms.
d. a method for protein expression prediction using machine learning.

2. The method of claim 1, wherein the host organism includes but is not limited to prokaryotic species.

3. The method of claim 2, wherein the host organism is a strain from the genera Escherichia-Shigella, Bacillus, or Corynebacterium.

4. The method of claim 1, where the host organism is a known eukaryotic organism.

5. The method of claim 4, where the host organism is a strain from the genera Trichoderma, Pichia, Saccharomyces, and Aspergillus.

6. The method of claim 1, wherein the protein length is 1,022 amino acids or less.

7. The method of claim 1, where the input sequence is a DNA sequence.

8. The method of claim 1, where the input sequence is a protein sequence.

9. The method of claim 1, wherein the predictive modeling technique preprocesses a mRNA sequence into a tensor for subsequent analysis using an architecture beginning with a UNet module followed by convolutional and fully-connected layers.

10. The method of claim 1, wherein the logits-based algorithm employs logits derived from advanced probabilistic models, including but not limited to the ProteinMPNN and ESM model family, guiding the exploration of the sequence space and enabling efficient identification of beneficial mutations.

11. A computational platform for optimizing protein expression and generating protein variants, comprising:
a. a system capable of processing and analyzing biological data to produce optimized genetic constructs and operate on standard and advanced computing hardware.
b. an input reception subsystem for receiving input information derived from DNA or protein sequences and configuration information.
c. a data processing engine subsystem using RNA spatial models for expression prediction and species-specific protein abundance models, utilizing amino acid sequences and incorporating machine learning and evolutionary algorithms for sequence optimization.

12. The method of claim 11, wherein the host organism includes but is not limited to prokaryotic species.

13. The method of claim 12, wherein the host organism is a strain from the genera Escherichia-Shigella, Bacillus, or Corynebacterium.

14. The method of claim 11, where the host organism is a known eukaryotic organism.

15. The method of claim 14, where the host organism is a strain from the genera Trichoderma, Pichia, Saccharomyces, and Aspergillus.

16. The method of claim 11, wherein the protein length is 1,022 amino acids or less.

17. The method of claim 11, where the input sequence is a DNA sequence.

18. The method of claim 11, where the input sequence is a protein sequence.

19. The computational platform of claim 11, further configured to process inputs for hundreds of sequences per second and capable of scaling for parallel processing to accommodate various levels of sequence throughput.

20. A method for empirical validation of protein expression predictions, comprising:
a. Correlating predicted protein expression values with experimental values from recombinantly expressed proteins.
b. Utilizing a comprehensive dataset comprising variant GFP sequences for training and evaluating the predictive models, ensuring robustness and reliability in predictive capabilities.
